(19) United States
(12) Patent Application Publication
(10) Pub. No.: US 2016/0166161 A1
(43) Pub. Date: Jun. 16, 2016

(54) Novel Design Considerations in the Development of a Photoplethysmography Ring

(71) Applicants: Bruce Yang, Irvine, CA (US); Tyler Bywaters Rice, Irvine, CA (US); Sean White, Irvine, CA (US)

(72) Inventors: Bruce Yang, Irvine, CA (US); Tyler Bywaters Rice, Irvine, CA (US); Sean White, Irvine, CA (US)

(21) Appl. No.: 14/879,603

(22) Filed: Oct. 9, 2015

Related U.S. Application Data
(60) Provisional application No. 62/061,701, filed on Oct. 9, 2014.

(51) Int. Cl. A61B 5/024; A61B 5/00

(52) U.S. Cl. CPC A61B 5/02427; A61B 5/02438; A61B 5/6826; A61B 5/6829

(57) ABSTRACT

An apparatus for measuring vital signs and various states of health using the underlying concept of Photoplethysmography (PPG). This apparatus comprises one or more light source(s) and one or more detector(s) configured to measure transmitted and/or reflected light within the digits such as a finger or toe, but can also be applied to other soft tissue elements. The apparatus comprises a means to enhance signal quality, control the volume of interrogated tissue and minimize motion artifact. The apparatus comprises a power source. The apparatus may further comprise at least one processor to receive information from the detector and process measured variations in light intensity. This information may be transmitted wirelessly through technology such as Bluetooth. The apparatus may also comprise a system utilizing inductive charging (“wireless charging”).

### CROSS-REFERENCE TO RELATED APPLICATIONS

This application claims priority from U.S. Provisional No. 62/061,701, filed Oct. 9, 2014, which is incorporated herein by reference and should be considered a part of this specification.

### BACKGROUND

1. Field

Disclosed herein are novel design considerations for improving the functionality of a wearable PPG ring. More particularly: improved signal quality by probing desired volumes of the sample more directly, while also minimizing motion artifact.

2. Description of the Related Art

Medical monitoring has moved into a new era as technological advances have made substantial improvements to the size and energy requirements of commonly used components. As such, ideas that were previously deemed impractical are becoming increasingly feasible. The focus of this patent is a novel approach at addressing problems that made a previously described invention unusable due to its impracticality.

Photoplethysmography (PPG) is an optical technology often used to monitor volumetric changes within an organ. PPG is typically performed by using a light source to project light into the organ/tissue of interest while simultaneously measuring the intensity of this light using an optical detector. Common embodiments utilize a light emitting diode (LED) to project light into the tip of the finger and use a photodiode placed next to (reflectance detection) or on the opposing side of the LED (transmission detection) for light detection. With proper choice of light wavelength and geometry, fluctuations in the amount of detected light can be related to volumetric changes. One of its most common uses is measure changes in blood volume within a tissue of interest to assess information such as heart rate. Heart rate is one of the most important parameters used to assess a subject's physical state. The ability to noninvasively monitor heart rate offers valuable medical information, and the ability to do so frequently could prove to be very insightful in the assessment of a subject's physical well-being. The frequency of measurements may depend on the needs of the user, and may vary anywhere from non-stop continuous monitoring, to several brief measurements per day, for example.

In order to implement daily, continuous heart monitoring, we describe a PPG device fabricated into a form factor many people often wear continuously—a ring. This concept of a PPG ring was initially developed in 1996 by a group at Massachusetts Institute of Technology. This initial concept, although functional, was impractical for adoption into mainstream sale and usage due to its obtrusive size, unreliable functionality, and lack of aesthetic appeal. The collaborators on this initial project were likely cognizant of, these shortcomings and therefore continued to make improvements. In 2000, Boo-Ho Yang and Sokwoo Rhee published an update to their initial ring which also included a conceptual drawing.

Despite these shortcomings, which can now be addressed due to advances in modern technology (such as miniaturized and more power efficient components), we believe two additional key problems prohibit the development of a practical functional PPG ring: 1) it is difficult to maintain consistent contact between the source and/or sensor with the tissue of interest, and 2) the chosen design must provide adequate comfort and wearability. These problems are critical toward developing a PPG ring that provides high-fidelity data while maintaining commercial appeal to the general public. This patent describes a method by which a PPG ring can, be made to satisfy the aforementioned criteria.

### SUMMARY OF THE INVENTION

An apparatus utilizing the principles of PPG for the development of a wearable ring for the purpose of health monitoring. This apparatus comprises a light source or a plurality of light sources and a detector or a plurality of detectors configured to measure transmitted and/or reflected light within the digits such as a finger or toe, but can may also be applied to other soft tissue elements. The apparatus comprises a means to enhance signal quality through more direct interrogation of the subject/area being probed while also minimizing motion artifact. The apparatus will accomplish the aforementioned objectives by incorporating source and/or detector elements into what jeweler's refer to as sizing beads; henceforth referred to as “signal protrusions” because they have the ability to enhance source delivery of light to the sensing detector(s), which translates into greater “signal” fidelity. The apparatus may further comprise at least one processor to receive information from the detector and process measured variations in light intensity. This information may further be transmitted wirelessly through wireless technology such as Bluetooth. The apparatus may also comprise a system utilizing inductive charging “wireless charging”.

### BRIEF DESCRIPTIONS OF THE DRAWINGS

FIG. 1 illustrates one embodiment of the invention showing the band of a ring with signal protrusions.

FIG. 2 illustrates one embodiment of the ring and signal protrusions on a finger.

FIG. 3 a-d. illustrates possible configurations and combinations of source and detector elements within the band of a ring.

FIG. 4 illustrates one embodiment of signal protrusions located on the band of a ring in two different views.

FIG. 5 illustrates one embodiment where source and detector signal protrusions are integrated as one protrusion.

FIG. 6 illustrates a blown-up view of source and detector signal protrusions in conjunction with soft tissue displacement in between the protrusions.

FIG. 7 illustrates a blown-up view of a single signal protrusion, in this case a source signal protrusion, in conjunction with a traditional embedded detector.

FIG. 8 illustrates a blown-up view of a combined source/detector signal protrusion into the soft tissue.

### DETAILED DESCRIPTION

An apparatus utilizing the principles of PPG for the development of a wearable ring for the purpose of health monitoring, utilizing the principle of sizing beads.within the ring to enhance signal fidelity and reduce motion artifact.

The concept of a PPG ring is was introduced prior to this application, but previous design concepts required improvement for practical mainstream adoption. Some of the problems previously described may be solved by adapting concepts used by jewelers to size rings and improve ring comfort, wearability, and fit. Commonly known as sizing beads, bishop beads, anti-roll beads, etc., these features improve ring comfort and sizing in a number of situations such as arthritic or swollen hands, rings that might be hard to size through other methods, rings rotating about the finger, etc. Sizing beads are typically two rigid or semi-rigid beads/mounds placed within the inner side of a ring as a means Of tightening the fit of the ring around the finger without adjusting the inner diameter (size) of the ring. While sizing beads are indeed oftentimes approximately hemispherical, thus akin to beads, the more important concept of the bead is the feature of physical protrusion from the band of the ring. Placing the light source and/or detector into or proximal to optically transmissive sizing protrusions, referred to as signal protrusions, alleviates the problems of maintaining contact of the source and/or sensor with the tissue of interest and providing adequate comfort and wearability. By incorporating the PPG light source and/or detector into protrusions, their consistent contact with the skin of the finger is greatly improved; this is due to the fact that the signal protrusions can be designed such that they protrude far enough from the inner surface of the ring that they will maintain contact with the skin of the tissue even if the entire inner side of the ring does not maintain consistent contact. Signal protrusions also reduce motion artifact due to ring movement about the finger.

The source and/or detector of the signal protrusion does not need to be wholly incorporated into the protrusion. Effective signal protrusion design can also result from protrusions being made of materials that can channel/direct, source/detector signals through the protrusions to source and/or detector elements located in the band. The incorporation of source and/or detector into signal protrusions mainly capitalizes on protrusions from the band of the ring to more effectively deliver and detect light information. As previously implied, it may be the case that only one component, the source or the detector, needs to be incorporated into the signal protrusion element or elements, and the other source or detector element incorporated into the inner diameter of the band, like previous PPG rings, where both these elements were simply incorporated into what would be considered the band component of the ring described herein.

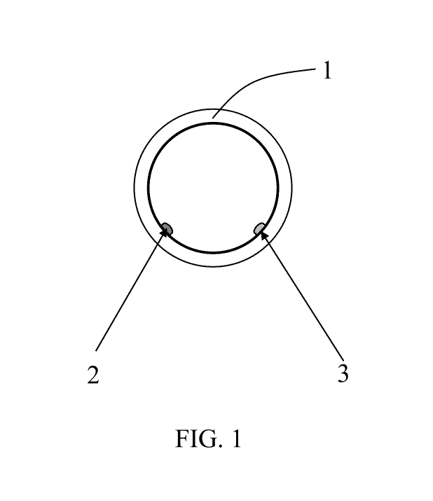

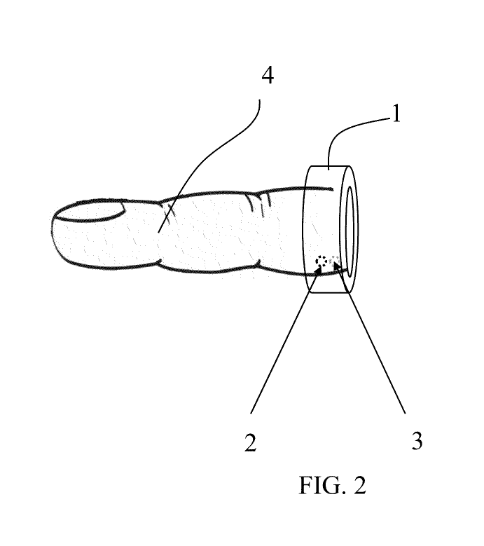

Referring to FIGS. 1 and 2, one embodiment of the signal protrusions in the band of a ring where a source signal protrusion 2 is located in the approximately 7:30 region of the band 1, if the ring of the band was referenced like the tick marks in a clock, and a detector signal protrusion 3 located in approximately the 4:30 region. This is just one possible embodiment of the apparatus of placement of the signal protrusions 2, 3 within the band 1, The location and combination of multiple signal protrusions 2, 3 can be varied to improve signal fidelity. FIG. 2 illustrates one embodiment of the ring as it might appear on a finger 4.

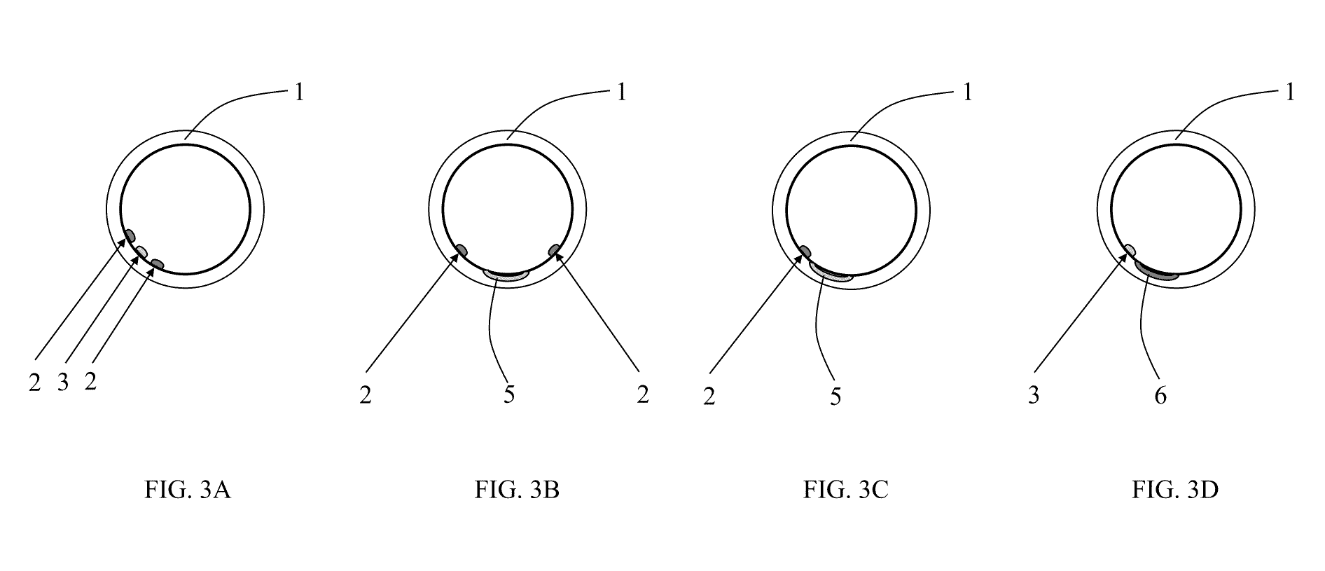

Referring to FIGS. 3 a-d, there can be significant variation in how these signal protrusions 2, 3 can be localized and utilized. FIGS. 3 a-d. illustrate some possible embodiments of signal protrusions 2,3 as well as how they can be arranged within the band along with currently used embedded detectors 5, and currently embedded sources 6.

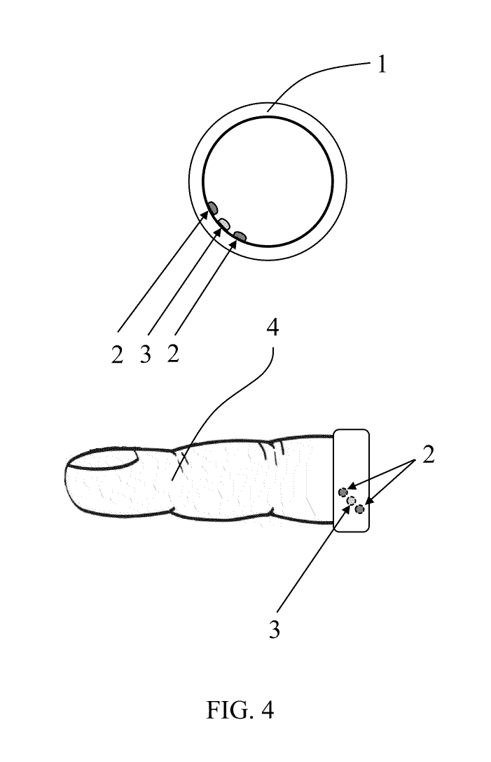

FIG. 4 illustrates how signal protrusions do not have to be axially aligned on a singular axis of the band 1.

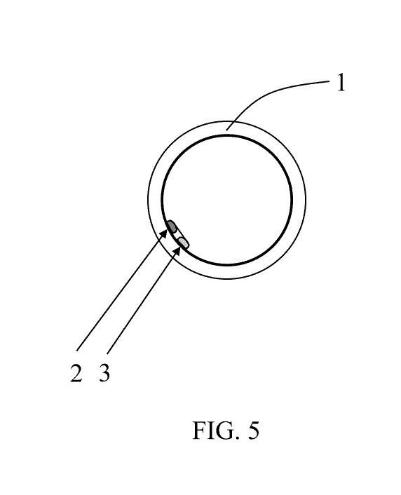

FIGS. 5,8 illustrates how signal protrusions 2, 3 for both source and detector can be combined together as one single protrusion.

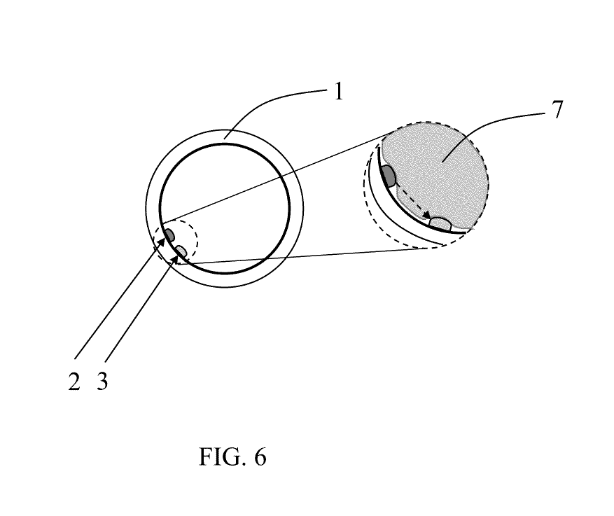

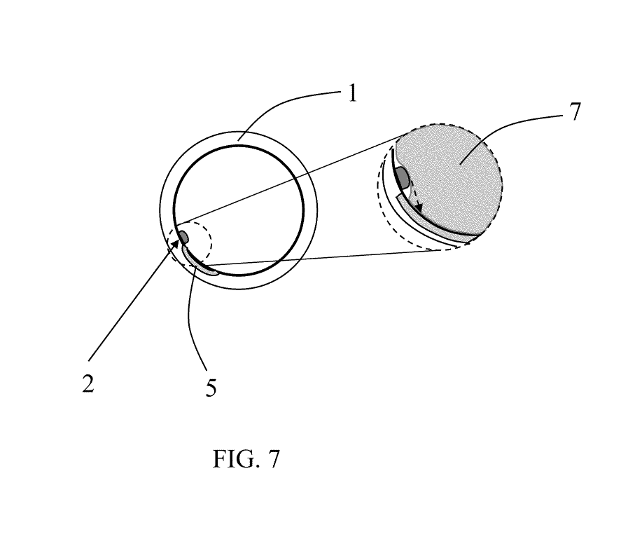

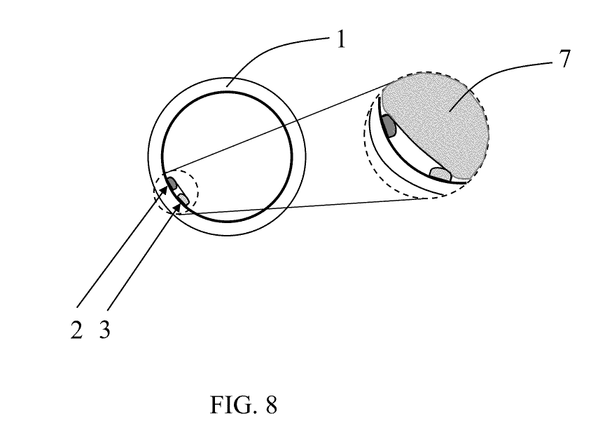

FIGS. 6-8 illustrate the potential significance of source and detector separation, whether it is between signal protrusions 2, 3 FIG. 6, a source signal protrusion 2 and a traditional detector 5 FIG. 7, and bow it compares to having both source and detector signal protrusions 2, 3 combined FIG. 8. FIGS. 6-8 illustrate different scenarios in how the soft tissue 7 of interest can interact with the different embodiments of the signal protrusions 2, 3 and traditional source/detector elements 5, 6. FIGS. 6, 7 demonstrate how soft tissue interaction between a source and detector can be enhanced when source and detector are not combined into one element as it is in FIG. 8. Furthermore, while we do illustrate possible advantages and disadvantages between a myriad of source and detector embodiments, we acknowledge the possible viability of any of these embodiments as adequate for the design of a PPG ring sensor.

A significant component of enhancing signal strength is the ability to have the source propagate through adequate volumes of soft tissue to be affected by physiological changes within the body; this can be intensified when the signal is allowed to pass through an unobstructed path between source and detector or by orienting source(s) and detector(s) to preferentially measure specific tissue volumes/features.

The protrusion/projection of the sizing protrusions into the tissue of the finger also reduces the ease with which the ring rotates about the finger, thus increasing comfort and wearability.

Wearablity is improved using this strategy because a proper ring fit can be achieved using sizing beads without needing to make the ring uncomfortably tight in the effort to maintain its constant contact with the skin of the finger. This novel application of incorporating PPG source and sensor elements into sizing beads within a ring solves two of the most significant problems seen in the previous PPG ring designs.

Since the signal protrusions 2, 3 will serve multiple functional roles beyond their initial design origin of sizing beads, anticipated design considerations include a wide possible range of choices for material from soft silicone, rigid light transmitting plastic, to rigid metal housing. Signal protrusions 2, 3, can also be designed to be spring-loaded to further improve contact to tissue while minimizing discomfort.

Furthermore, variations in possible signal protrusion 2, 3 placement, may benefit from different mechanisms for re-localization and removal such as screwing, sliding along a track, snapping into place, and magnetism/ Signal protrusion 2, 3 adjustability can-also be a key element to accommodate the wide variation of finger/toe variations seen in the population or just even changes seen within the same subject due to weight gain or weight loss. Variable location of the signal protrusions may also aid in improving signal fidelity as different source-detector orientations acquire information from varying tissue volumes, some of which may provide more informative or higher-fidelity data.

Additional considerations to maximize usability include data transfer, power, charging, visualization/display, alerts, and ring housing considerations.

Data Transfer

Data transfer can take place by either being hard-connected/wired into a separate device including, but not limited to, a watch or phone. Alternatively data transfer can take place wirelessly via, for example, Bluetooth. Due to size limitations, being connected to a device such as a watch via a wired or wireless connection can offer obvious benefits such as power, data processing, and a display to view information. However, all of these features can be contained within in the ring itself as a standalone device as well.

Power

Power for the PPG ring can come in various forms, including but not limited to one or more, or a combination of, the following: 1) internal battery 2) external battery 3) solar/thermal 4) kinetic energy 5) chemical energy 6) induction. The external battery may be easily connected to and removed from the ring. One possible embodiment is an external battery designed to resemble the commonly seen precious stone. This could allow the user to have on-band multiple batteries to quickly switch out.

Charging

Charging the battery may take place in both a wired/hard-connected fashion and a wireless fashion. Wireless charging for example can be through induction. Charging may also be achieved using conductive contacts on the ring which are oriented to mate with corresponding charging unit.

Visualization/Display

The ring itself can have a display to show the users such parameters as heart rate, respiratory rate, etc. This information may also appear on a separate device such as a watch or phone.

Alerts

With one or multiple physiological parameters being measured, the PPG ring may provide alerts to the user stemming from raw or processed data. For example, during exercise, the ring may use a display, audible signal, or haptic feedback to alter the user about the value of specific parameters, such as their heart rate. An additional example is the ring using a display, audible signal, or haptic feedback to inform the user that their respiratory rate, which Can be derived from the PPG signal, has dropped below a set threshold or has become irregular.

Housing Considerations

A practical and usable PPG ring requires new and novel design considerations regarding the housing, or casing, of the ring. One design consideration includes a ring whose internal parts are confined/molded within a material such as silicone. Silicone rings have become quite popular among active people who prefer a more soft and malleable material. Silicone rings may also allow more consistent contact between the source and/or and the compared to a more rigid ring. Another possibility is separate parts joined together to form the housing. For example, two halves of a ring can be joined together through a snap mechanism, a screw mechanism, using an elastic band, etc. This allows for the ability to take apart the ring to access different parts for possible modification or repair. It may also allow for finer adjustment of ring fit than a typical rigid ring, thus improving consistent contact between the source and/or detector and the skin of the finger, as well as comfort and wearability.

REFERENCES Herein Incorporated by Reference

1. Yang, Boo-Ho, and Sokwoo Rhee. “Development of the ring sensor for healthcare automation.” Robotics and Autonomous Systems 30.3 (2000): 273-281 .

What is claimed is:

1. An apparatus for health monitoring, comprising:
a light source; and
a detector configured to receive transmitted and/or reflected light from a tissue sample,
wherein the source and/or detector are incorporated into protrusions located within a ring-like structure.

2. The apparatus of claim 1, wherein the source or sources is/are incorporated in a protrusion from the band of the ring.

3. The apparatus of claim 1, wherein the detector or detectors is/arc incorporated in a protrusion from the band of the ring.

4. The apparatus of claim 1, wherein the source or sources is/are located within the band of the ring and light is transmitted through a material that protrudes from the band of the ring.

5. The apparatus of claim 1, wherein the detector or detectors is/are located in the band of the ring and a material that protrudes from the ring is capable of receiving a signal.

6. The apparatus of claim 1, wherein the source(s) and detector(s) are incorporated into a combined protrusion from the band of the ring.

7. The apparatus of claim 1, wherein the apparatus comprises a power source.

8. The apparatus of claim 1, wherein the apparatus comprises a processor.

9. The apparatus of claim 1, wherein the tissue sample is a finger.

10. The apparatus of claim 1, wherein the tissue sample is a toe.

11. The apparatus of claim 1, wherein the power source is charged using induction.
